(19) United States
(12) Patent Application Publication
(10) Pub. No.: US 2023/0338272 A1
(43) Pub. Date: Oct. 26, 2023

(54) Process for Transfer Resistance Polymers in Pigmented Products

(71) Applicant: Thomas O'Lenick, Monroe, GA (US)

(72) Inventor: Thomas O'Lenick, Monroe, GA (US)

(21) Appl. No.: 18/303,138

(22) Filed: Apr. 19, 2023

Related U.S. Application Data
(60) Provisional application No. 63/332,842, filed on Apr. 20, 2022.

(51) Int. Cl. A61K 8/86; A61Q 1/06

(52) U.S. Cl. CPC A61K 8/86; A61Q 1/06

(57) ABSTRACT

The present invention disclosed herein relates to a process for minimizing the transfer of lipstick from lips. The process for imparting transfer resistance involves a solvating a polymer in volatile solvent and applying to the lips. The application of the polymer solution can be incorporated as a topcoat over a commercial lip product. The polymer creates a hydrophobic film that prevents transfer of the lipstick underneath the film and prevents wash off as a result from contact with water.

### RELATED APPLICATIONS

This patent claims priority over provisional patent U.S. 63/332,842 filed on Apr. 20, 2022.

### GOVERNMENT SPONSORSHIP

None

### FIELD OF THE INVENTION

Cosmetic compositions are generally defined as compositions suitable for application to the human body. Cosmetic compositions such as creams and lotions are used to moisturize skin and keep it in a smooth supple condition. Pigmented cosmetic compositions such as makeup, blush, lipstick, etc. are used color to the skin and lips. Since color is one of the most important reasons for wearing cosmetics, color containing cosmetics must be very carefully formulated to provide maximum wear and effect.

The present invention is directed to cosmetic compositions that are silicone free, naturally derived and provide transfer resistance to pigmented products applied as cosmetics to the skin. The compositions of the present invention are pigmented products that have a cosmetically elegant feel and provide both oil resistance and water resistance easily applied to the skin. Once applied they do not transfer to ceramic cups, skin, paper or textile fabric. The polymer that replaces the silicone in this cosmetic application is disclosed in a patent U.S. Pat. No. 8,465,730 issued Jun. 18, 2013, to Thomas O'Lenick entitled polyglycerol polyesters. incorporated herein by reference. All patents referenced herein are incorporated by reference to the extent allowed by law.

### BACKGROUND OF THE INVENTION

There has been a long felt need for a transfer pigmented product for use as a cosmetic since ancient times. Ancient Sumerian and Indus Valley men and women were possibly the first to invent and wear lipstick, about 5,000 years ago. Sumerians crushed gemstones and used them to decorate their faces, mainly on the lips and around the eyes. Egyptians like Cleopatra crushed bugs (carmine) to create a color of red on their lips. Women in the ancient Indus Valley Civilization have used rectangular pieces of ochre with beveled ends as lipstick. The Kamasutra describes lip coloring made of red lac and beeswax and method it was used. Ancient Egyptians wore lipstick to show social status rather than gender. They extracted the red dye from fucus-algin, 0.01% iodine, and some bromine mannite, but this dye resulted in serious illness. Lipsticks with shimmering effects were initially made using a pearlescent substance found in fish scales.

The application of pigment, applied to skin to beautify the skin needs to have a number of attributes that make it attractive to the consumer. These attributes include but are not limited to ease of application, spreadability on the lips, comfort once applied to the lips, remaining on the lips and not transferring to other surfaces like cups, and clothing. The lipstick must be flexible not peeling off due to differences in the flexibility of the lipstick and the lips. Finally, once they remain on the skin for the desired period of time, they must be easily and cleanly removed from the lips. Recently, there has been a strong desire to formulate cosmetic products with natural or naturally derived ingredients that will biodegrade and be harmless in the environment. These latter requirements have made many traditional ingredients that are not environmentally friendly in lipstick formulations being replaced with raw materials having a better environmental profile.

In the mid to late 90s, complex silicones including dilauroyl trimethylolpropane siloxy silicate or diisostearoyl trimethylolpropane siloxy silicate enabled transfer-resistant properties in Revlon's lipsticks. U.S. Pat. No. 5,505,937, Cosmetic compositions with improved transfer resistance, A Castrogiovanni et al, assigned to Revlon Consumer Products Corp (April 1996).

These were introduced to the formulations as mixtures with volatile oils. The volatile oils aided in spreading the compositions on skin. Upon contact, they evaporated, and the transfer resistant properties and durable wear of the formulas were then realized. It should be noted that in conjunction, packaging was designed to prevent the evaporation of the volatile oil from the product during storage and between U.S.es. With this in mind, educating the consumer to snap the cap shut after use was required to prevent the product from drying into a hard glob that would not “pay out.”

Revlon demonstrated the transfer resistance of its Ultima II formulation using a “Kiss Test.” Panelists were instructed to apply the lipstick, wait five minutes to allow the volatile oil to evaporate, and then kiss the back of their hand. A resulting 87% of panelists reported the lipstick left “hardly a trace” of color on their hand. Since then, the Kiss Test has become a standard to evaluate the propensity of a lipstick to transfer.

In 1994, U.S. Pat. No. 5,330,747 entitled Cosmetics with enhanced durability issued to Duane Kryzsik disclosed that pressure-sensitive silicone adhesives improved adherence, and he exemplified this in mascara formulations that resisted wash-off. These pressure-sensitive adhesives included a trimethylsilyl end-blocked resin, a silanol terminated dimethicone and a phenyltrimethicone.

U.S. Pat. No. 5,800,816, entitled Cosmetic compositions, issued to H Brieva, JG Russ and IM Sandewicz, assigned to Revlon Consumer Products Corp (Sep. 1, 1998) and U.S. Pat. No. 5,911,974, H Brieva, J G Russ and I M Sandewicz, assigned to Revlon Consumer Products Corp (Jun. 15, 1999) teach that trimethylated silica in lipstick confers transfer-resistant properties to film deposited on the lips. U.S. Pat. No. 5,911,974, H Brieva, J Russ and I M Sandewicz, assigned to Revlon Consumer Products Corp (Jun. 15, 1999) teaches that trimethylated silica initially prepared by dispersing fine-grained silica in a solvent mixture of low-viscosity silicone oil in a volatile hydrocarbon. When the volatile hydrocarbon was distilled away, a smooth and transparent paste or grease was obtained. This inspired Revlon scientists to explore the possibility of Using such volatile/involatile mixtures in makeup products. The volatile silicones enabled product spreading onto the skin or lips and once dried, the trimethylated silica conferred transfer resistance. use of volatile solvents that evaporate after application were evaluated and several patents were issued most notably:
U.S. Pat. No. 5,800,816, Cosmetic compositions, H Brieva, J G Russ and I M Sandewicz, assigned to Revlon Consumer Products Corp (Sep. 1, 1998);
U.S. Pat. No. 6,274,152, Cosmetic compositions, H Brieva, J G Russ and I M Sandewicz, assigned to Revlon Consumer Products Corp. (Aug. 14, 2001);
U.S. Pat. No. 6,464,964, Cosmetic compositions, H Brieva, J G Russ and I M Sandewicz, assigned to Revlon Consumer Products Corp. (Oct. 15, 2002)
U.S. Pat. No. 6,562,322, Cosmetic compositions, H Brieva, J G Russ and I M Sandewicz, assigned to Revlon Consumer Products Corp. (May 13, 2003)
U.S. Pat. No. 5,800,816, Cosmetic compositions, H Brieva, J G Russ and I M Sandewicz, assigned to Revlon Consumer Products Corp (Sep. 1, 1998).

This technology was extended to mascaras, concealers, foundations, eye shadows and sunscreens by incorporating film-forming polyamide or acrylate copolymers.¹⁷ Shiseido, Procter & Gamble and Revlon researchers all have pursued the use of silicone resins to attain transfer-resistant properties. Such compounds are made up of units generally described as M, D, T and Q monomer units.

U.S. Pat. No. 5,676,938, entitled Cosmetic composition, issued to H Kimura, Y Horei and Y Sato, assigned to Toshiba Silicone Co (Oct. 14, 1997) teaches that organopolysilsesquioxanes of the MQ type conferred good water-repellency and long wear in cosmetic compositions.

However, the resins were soluble in the same phase as cosmetic pigments, and oil in that phase caused swelling and discontinuity of the silicone films of such resins on the skin. As a consequence, the pigment became detached, and the color ran. U.S. Pat. No. 5,948,393, Make-up cosmetic composition, S Tomomasa, H Takada and Y Soyama, assigned to Shiseido (Sep. 7, 1999)

Japanese Patent Application 61-158913, to Shiseido (Jul. 18, 1986) discloses an improved the water-repellency and conferred oil-resistance to the cosmetics by replacing the oils with volatile silicones and volatile oils.

Japanese Patent Application 61-158913, Shiseido (Jul. 18, 1986) discloses a liquid lip makeup that was non-transferable to surfaces such as drinking glasses. This composition contained 40% MQ resin, 20% volatile hydrocarbon oil, 20% powder, 10% glyceryl tri-isostearate and 10% red dye.

A series of discloses 6 patents disclose a transfer-resistant lipstick comprising MQ resin, dimethicone gum, isododecane or cyclomethicone as the volatile carrier, and pigments for color. This is interesting for two reasons: one, the formulation limits were defined graphically, with respect to silicone fluid viscosity, polymer/pigment and organosilicone resin/silicone fluid concentrations and second, the compositions were tested for transfer resistance by dry blot and rub, and oil blot and rub by applying the compositions to collagen sausage casings and spectroscopically measuring the transfer (or lack thereof) when the collagen was contacted with a foam plate. The patents are;
U.S. Pat. No. 6,071,503, Transfer resistant cosmetic compositions, L E Drechsler, T Rabe, T E Smith and E Dewey, assigned to Procter & Gamble (Jun. 6, 2000)
U.S. Pat. No. 6,071,503, Transfer resistant cosmetic compositions, L E Drechsler, T Rabe, T E Smith and E Dewey, assigned to Procter & Gamble (Jun. 6, 2000)
U.S. Pat. No. 6,074,654, LE Drechsler, T Rabe, T E Smith and E Dewey, assigned to Procter & Gamble (Jun. 13, 2000)
U.S. Pat. No. 6,139,823, LE Drechsler, T Rabe, T E Smith and E Dewey, assigned to Procter & Gamble (Oct. 31, 2000)
U.S. Pat. No. 6,340,466, LE Drechsler, T Rabe, T E Smith and E Dewey, assigned to Procter & Gamble (Jan. 22, 2001)
U.S. Pat. No. 6,406,683, LE Drechsler, T Rabe, T E Smith and E Dewey, assigned to Procter & Gamble (Jun. 18, 2002)

Although durability and transfer-resistance are desired in a lipstick, it should be easily removed when desired. Accordingly, Procter & Gamble inventors promoted their transfer-resistant lipsticks as easily removed by applying petrolatum or a dimethicone-based cosmetic remover. Thereafter, specific removers were developed for the removal of transfer-resistant cosmetics.

U.S. Pat. No. 7,404,966, Transfer-resistant makeup removing compositions, M L Vatter, assigned to Procter & Gamble (Jul. 29, 2008) disclosed one such remover comprised cross-linked silicone elastomers and a solvent suitable for application to skin. In this case, the cross-linked siloxanes were dimethicone/vinyl dimethicone crosspolymers with particles sizes 10-200μ. Particle sizes below 10μ reduced the skin feel and viscosity; above 200μ, silicon elastomer particles formed gel balls on the skin.

C D Vaughan, Solubility effects in product. Package, penetration and preservation, Cosm & Toil (103) 47-69 (October 1988) and CD Vaughan, using solubility parameters in cosmetics formulation, J Soc Cos Chem (36) 319-333 (September/October 1988) taught that the preferred liquid carriers were those having solubility parameters of less than ˜9.

U.S. Pat. No. 8,241,617, Methods for removing make-up compositions from keratin materials, M Chen, V Chou and P McDermott, assigned to L'Oréal (Aug. 14, 2012) discloses a removal composition based on nylon-611/dimethicone copolymer, and dimethicone or phenylmethicone.

U.S. Pat. No. 6,033,650, Glossy transfer resistant cosmetic compositions, J F Calello, A A Patil, S J Barone and A M Krog, assigned to Revlon Consumer Products Corp (Mar. 7, 2000) discloses the model of MQ resin plus oil was essentially a “film-former plus plasticizer” combination. Since MQ resin essentially is a tackifier, formulators were forced to compromise between high tack with good skin adhesion, and low tack with poor adhesion. Moreover, the original transfer-resistant lipsticks bestowed a dry feel and matte finish to the lips—although at that time, marketing studies revealed that three out of five women preferred glossy lipstick because it conveyed youthfulness and good health.

Transfer-resistant lipsticks with reduced permeability to oil and water also were sought so that consumers could eat, drink and even blot away perspiration or exuded sebum without losing their made-up appearance. U.S. Pat. No. 5,985,298, Cosmetic compositions, H R Brieva, J G Russ and I M Sandevicz, assigned to Revlon Consumer Products Corp (Nov. 16, 1999) and U.S. Pat. No. 7,005,134, Cosmetic compositions, H R Brieva, J G Russ and I M Sandevicz, assigned to Revlon Consumer Products Corp (Feb. 28, 2006) teach that this was achieved by incorporating perfluoropolyether liquids in lipstick to act as a barrier against its dissolution from the lips. The unique properties of polyfluorocarbons arise from the strength of their C—F bonds, which at 485 Kilojoules/mole (Kj/m), are much stronger than C—H (413 Kj/m) or C—C bonds (348 Kj/m). When all the hydrogen atoms of a hydrocarbon chain are replaced by fluorine atoms, the molecule is effectively covered in an impermeable sheath that resists acids, alkalies, enzymes, oils, solvents and aqueous. solutions—hot or cold. Polyperfluoromethylisopropyl ether also has a very low surface tension, which enables it to spread quickly and evenly over surfaces. Additional advantages include the material being non-greasy and not easily flushed from the skin by perspiration. It also does not impregnate silicones or other polymers, and it cannot wet an adsorbed layer of itself—rather, it forms a thin coating, often a monolayer, over other materials to render them non-wettable by most common liquids.

One of the long standing problems with makeup, particularly lipstick, is the tendency of the cosmetic to blot or transfer from the skin onto other surfaces such as glassware, silverware, clothing, etc. This not only creates soiling on implements, but forces the lipstick user to reapply the cosmetic at fairly short intervals in order to keep the lips colored. U.S. Pat. No. 11,160,735 to Martinez issue Nov. 2, 2021, entitled Long wear lip cosmetic system and topcoat is the most recent patent on this topic. It clearly requires silicone polymers and very different from the technology used in the current invention. '735 clearly states “According to preferred embodiments, the long-wear lip compositions of the present invention include at least one silicone resin. Examples of suitable silicone resins include those described, for example, in U.S. Pat. Nos. 5,505,937, 5,911,974, 5,965,112, 5,985,298, 6,074,654, 6,780,422, 6,908,621, the disclosures of which are hereby incorporated by reference in their entirety”.

“Film former” or “film forming agent” or “film forming polymer” or “film forming resin” as used herein mean a polymer or resin that leaves a film on the substrate to which it is applied, for example, after a solvent accompanying the film former has evaporated, absorbed into and/or dissipated on the substrate.

“Transfer resistance” as used herein refers to the quality exhibited by compositions that are not readily removed by contact with another material, such as, for example, a glass, an item of clothing or the skin, for example, when eating or drinking. Transfer resistance may be evaluated by any method known in the art for evaluating such. For example, transfer resistance of a composition may be evaluated by a “kiss” test. The “kiss” test may involve application of the composition to human keratin material such as hair, skin or lips followed by rubbing a material, for example, a sheet of paper, against the hair, skin or lips after expiration of a certain amount of time following application, such as 2 minutes after application. Similarly, transfer resistance of a composition may be evaluated by the amount of product transferred from a wearer to any other substrate, such as transfer from the hair, skin or lips of an individual to a collar when putting on clothing after the expiration of a certain amount of time following application of the composition to the hair, skin or lips. The amount of composition transferred to the substrate (e.g., collar, or paper) may then be evaluated and compared. For example, a composition may be transfer resistant if a majority of the product is left on the wearer's hair, skin or lips. Further, the amount transferred may be compared with that transferred by other compositions, such as commercially available compositions. In a preferred embodiment of the present invention, little or no composition is transferred to the substrate from the hair, skin or lips.

“Long wear” compositions as used herein, refers to compositions where color remains the same or substantially the same as at the time of application, as viewed by the naked eye, after an extended period of time. Long wear properties may be evaluated by any method known in the art for evaluating such properties. For example, long wear may be evaluated by a test involving the application of a composition to human hair, skin or lips and evaluating the color of the composition after an extended period of time. For example, the color of a composition may be evaluated immediately following application to hair, skin or lips and these characteristics may then be re-evaluated and compared after a certain amount of time. Further, these characteristics may be evaluated with respect to other compositions, such as commercially available compositions. For lip compositions, “long wear” typically means the composition remains on the lips at least about 4 hours up to about 24 hours, and retains rich color even after eating.

A lipstick topcoat typically consists of a non-volatile material capable of preventing transfer of a pigment from the lips. The topcoat composition of the present invention comprises a volatile solvent. Volatile solvents are capable of solubilizing polymeric materials and depositing them onto the lips from a clear solution. Non-limiting examples of volatile solvents according to an embodiment of the present invention include ethanol, isododecane, isohexadecane, propylene glycol n-butyl ether, ethyl 3-ethoxyproprionate, propylene glycol methyl ether acetate, isoparaffin C11-C13, and isoparaffin C11-C12.

“Liquid” or “liquid cosmetic” or “liquid lipstick” or “liquid composition” means a composition having a fixed volume, flows to cover the bottom and assumes the shape of the portion of the container it fills and is slightly compressible.

“Tackiness” as used herein refers to the adhesion between two substances. For example, the more tackiness there is between two substances, the more adhesion there is between the substances. To quantify “tackiness,” it is useful to determine the “work of adhesion” as defined by IUPAC associated with the two substances. Generally speaking, the work of adhesion measures the amount of work necessary to separate two substances. Thus, the greater the work of adhesion associated with two substances, the greater the adhesion there is between the substances, meaning the greater the tackiness is between the two substances.

Work of adhesion and, thus, tackiness, can be quantified using acceptable techniques and methods generally used to measure adhesion and the one far along described.

### THE INVENTION

### Objective of the Invention

It is the object of the current invention to impart transfer resistance to a pigmented cosmetic product by the incorporation of a naturally derived polyester. This product has functionality in pigmented products including but not limited to lipstick, mascara and topcoat products.

### SUMMARY OF THE INVENTION

The present invention relates to a process for imparting transfer resistance to pigmented cosmetic products which comprises contacting the lips with a product which comprises (1) at least one polyester described in U.S. Pat. No. 8,465,730 issued Jun. 18, 2013, and (2) a volatile carrier. The composition can provide transfer resistance as a topcoat, applying the composition over a cosmetic pigmented product, or the composition can be incorporated directly into the cosmetic pigmented product. Surprisingly and unexpectantly, unlike other formulations in order to obtain a transfer resistant film, only the compounds of the present invention and solvent are needed. This provides a two-component film former that can be added to pigment containing formulations.

### DETAILED DESCRIPTION OF THE INVENTION

One aspect of the current invention is a composition capable of preventing transfer of a pigmented cosmetic product which comprises:
1. from about 2.0 to 10.0% by weight of a polymer described in U.S. Pat. No. 6,456,730:

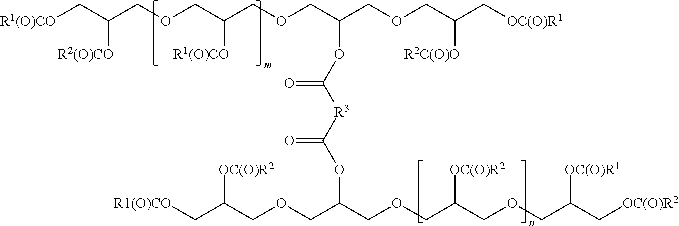

wherein,
R¹ is an alkyl containing 18 to 26 carbons atoms and is solid at 25° C.;
R² is an alkyl containing 8 to 22 carbons atoms and is liquid at 25° C.;
R³ is independently selected from an alkyl containing 7 carbon atoms or an alkyl confining to the following structure:

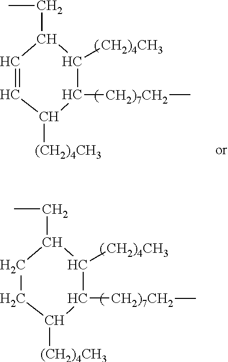

and mixtures thereof with the proviso that at least 50% of R³ is a solid at 25° C.
n is an integer ranging from 0 to 9;
m is an integer ranging from 0 to 9;
   and
2. from about 90-98% by weight of a solvent capable of forming a clear solution with the polymer;
The composition when applied as a topcoat or incorporated into a pigmented cosmetic product will prevent transfer.

### Preferred Embodiments

In a preferred embodiment the polymer conforms to the following structure:

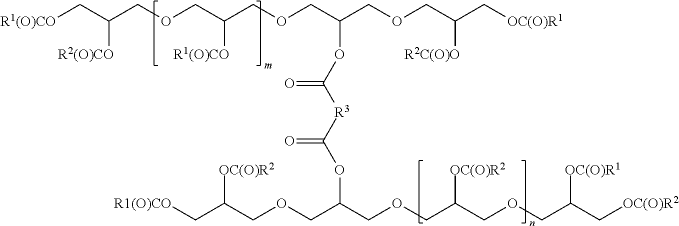

wherein,
R¹ is an alkyl containing 18 carbons atoms and is solid at 25° C.;
R² is an alkyl containing 18 carbons atoms and is liquid at 25° C.;
R³ is an alkyl containing 7 carbon atoms;
n is 0;
m is 0.

### EXAMPLES

### Raw Materials

### Solid Fatty Acids (R¹)

Fatty acids useful as raw materials in the preparation of compounds of the present invention are commercially available from a variety of sources including Procter and Gamble of Cincinnati Ohio. The structures are well known to those skilled in the art.

R—C(O)—OH

### Saturated

| Example | R Formula | Common Name | Molecular Weight |
|---|---|---|---|
| 1 | C₁₇H₃₅ | Stearic | 284 |
| 2 | C₂₁H₄₃ | Behenic | 340 |
| 3 | C₂₆H₅₃ | Cetrotic | 396 |
| 4 | C₃₃H₆₇ | Geddic acid | 508 |

### Liquid Fatty Acids (R²)

Fatty acids useful as raw materials in the preparation of compounds of the present invention are commercially available from a variety of sources including Procter and Gamble of Cincinnati Ohio. The structures are well known to those skilled in the art.

R—C(O)—OH

### Saturated

| Example | R | Common Name | Molecular Weight |
|---|---|---|---|
| 5 | C₇H₅ | Caprylic | 144 |
| 6 | C₉H₁₉ | Capric | 172 |
| 7 | C₁₇H₃₅ | Isosteric | 284 |
| 8 | C₁₇H₃₃ | Oleic | 282 |
| 9 | C₁₇H₃₂ | Linoleic | 280 |
| 10 | C₁₉H₃₉ | Arachidinic | 312 |
| 11 | C₂₁H₄₁ | Erucic | 338 |

### Example 12

### Dimer Acid

Dimer acid is an item of commerce available commercially from Cognis Corporation. It conforms to the following structure:

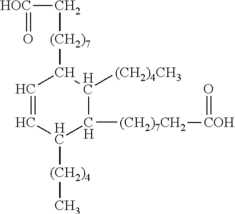

### Example 13

### Hydrogenated Dimer Acid

Hydrogenated dimer acid is an item of commerce available commercially from Henkel Corporation. It conforms to the following structure:

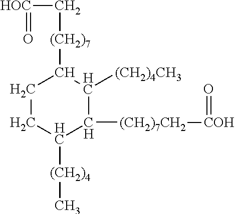

### Dicarboxylic Acids

Dicarboxylic acids useful as raw materials in the synthesis of the compounds of the present invention are commercially available from a variety of sources including Cognis. They conform to the following structure;

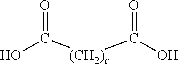

wherein;
c is an integer ranging from 1 to 10.

| | | | Molecular |
|---|---|---|---|
| Example | Common Name | c | Weight |
| 14 | Succinic | 2 | 118 |
| 15 | Azelaic | 7 | 188 |
| 16 | Sebacic | 8 | 202 |
| 17 | Dodecanedioic | 10 | 230 |

### Polyglycerol

Polyglycerol is useful as raw materials in the preparation of compounds of the present invention. Polyglycerols are commercially available from a variety of sources including Solvay Chemicals of Rheinberg Germany.

The structures are well known to those skilled in the art.

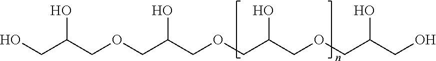

wherein;
n is an integer ranging from 0 to 9.

| Example | n | MW |
|---|---|---|
| 18 | 0 | 240.0 |
| 19 | 3 | 450.0 |
| 20 | 7 | 750.0 |

### Polyglycerol Polymers

Polyglycerol polymers are subjects of U.S. Pat. No. 6,465,730. They conform to the following structure:

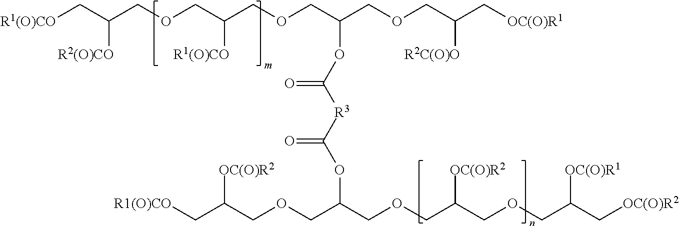

| | Polyglycerol | | R¹ | | R² | | R³ | |
|---|---|---|---|---|---|---|---|---|
| Ex- | Ex- | | Ex- | | Ex- | | Ex- | |
| ample | ample | Grams | ample | Grams | ample | Grams | ample | Grams |
| 21 | 18 | 68.01 | — | — | 6 | 84.61 | 15 | 53.06 |
| 22 | 18 | 68.01 | 1 | 84.61 | — | — | 15 | 53.06 |
| 23 | 18 | 74.59 | 1 | 92.73 | — | — | 13 | 37.59 |
| | | | | | | | 15 | 46.17 |
| 24 | 18 | 76.60 | 2 | 113.25 | — | — | 15 | 59.27 |
| 25 | 18 | 79.14 | 1 | 49.46 | 2 | 59.21 | 15 | 62.03 |
| 26 | 18 | 83.52 | 1 | 83.16 | 8 | 19.34 | 15 | 64.58 |

### Solvent

The composition includes at least one solvent. Typically, these solvents are volatile hydrocarbons. The term “solvent” as used herein, compounds having a melting point of less than about 25° C. at atmospheric pressure (1.013×10⁵ Pa) and are capable of forming a clear homogenous solution with the polymers of the current invention. Solvents utilized in the present invention include but are not limited to: ethanol, volatile hydrocarbon having from 8 to 16 carbon atoms and their mixtures and in particular branched C8 to C16 alkanes such as C8 to C16 isoalkanes (also known as isoparaffins), isododecane, isodecane, isohexadecane, and for example, the oils sold under the trade names of Isopar or Permethyl, the C8 to C16 branched esters such as isohexyl or isodecyl neopentanoate, alcohols, and their mixtures. Preferably, the solvent has a flash point of at least 40° C. Preferred solvents are found in table 1.

| | | Flash Point |
|---|---|---|
| Example | Solvent | (° C.) |
| 27 | Ethanol | 14 |
| 28 | Isododecane | 43 |
| 29 | Isohexadecane | 102 |
| 30 | Propylene glycol n-butyl ether | 60 |
| 31 | Ethyl 3-ethoxypropionate | 58 |
| 32 | Propylene glycol methylether acetate | 46 |
| 33 | Isoparaffin C₁₁-C₁₃ | 62 |
| 34 | Isoparaffin C₁₁-C₁₂ | 56 |

### Polymer Solutions

### Procedure

5.00 g of polyglycerol polymers (Examples 21-26) are placed into a beaker equipped with an overhead stirrer. 95.00 g of solvent (Examples 27-34) and the stirrer was stated. The mixture in the beaker was allowed to stir until a homogenous solution is produced.

| | |
|---|---|
| Polyglycerol Polymer | Solvent |

| Examples | Example | Grams | Example | Grams |
|---|---|---|---|---|
| 35 | 21 | 3.0 | 27 | 97.0 |
| 36 | 22 | 4.0 | 28 | 96.0 |
| 37 | 23 | 2.0 | 29 | 98.0 |
| 38 | 24 | 6.0 | 30 | 94.0 |
| 39 | 25 | 7.0 | 31 | 93.0 |
| 40 | 26 | 8.0 | 32 | 92.0 |
| 41 | 21 | 9.0 | 33 | 91.0 |
| 42 | 22 | 10.0 | 34 | 90.0 |
| 43 | 21 | 5.0 | 27 | 95.0 |
| 44 | 22 | 5.0 | 27 | 95.0 |
| 45 | 23 | 5.0 | 27 | 95.0 |
| 46 | 24 | 5.0 | 27 | 95.0 |
| 47 | 25 | 5.0 | 27 | 95.0 |
| 48 | 26 | 5.0 | 27 | 95.0 |

### Film Characteristics

In order to characterize the polymers' ability to be transfer resistance, a film was cast from ethanol and the water and oil contact angle were evaluated.

### BRIEF DESCRIPTION OF THE DRAWINGS

The transfer resistant properties of the current invention were tested by a transfer test. The results of this test are shown in the drawings. The better the performance, the less transfer will appear in the photo.

### DETAILED DESCRIPTION OF THE DRAWINGS

FIG. 1 shows an optical image of a contact angle of water on a lipstick formulation. The figure shows that the water contact angle is 18.59. The lipstick formulation is hydrophilic and will likely wash off when water is introduced. On the other hand, example 48 shows a contact angle of 96.40, which is hydrophobic and will resist rinse off by water.

FIG. 2 shows the transfer resistance test for the topcoat application of the transfer resistant composition. The images show a filter paper (bottom) and the cotton cloth (top). The amount of transfer is determined optically by the amount of pigment transferred to the cotton cloth.

FIG. 3 shows the transfer resistance test for the lipstick formulation. The images show a filter paper (bottom) and the cotton cloth (top). The amount of transfer is determined optically by the amount of pigment transferred to the cotton cloth.

### Transfer Resistance as a Topcoat

### Procedure

A 8.0-11.0 mg of lipstick was applied to a piece of filter paper. A solution of polyglycerol polymer (Examples 35-48) was applied on top of the filter paper and allowed to dry. After the topcoat dries, a piece of cotton cloth was put onto the filter paper. From the backside of the cotton cloth, a stress is applied for 30 seconds. The cotton cloth was then removed and visually inspected for pigment transfer.

### Lipstick formulation Example 54

| Ingredient | Amount |
|---|---|
| Castor Oil | 10.30 |
| Diisostearyl Malate | 15.00 |
| Phenyltrimethicone | 5.00 |
| Dioctyldodecyl Dimer Dilinolate Copolymer | 5.00 |
| CL 77891 | 4.00 |
| CL 77491 | 3.00 |
| CL 15850 | 1.00 |
| Octyldodecyl Riclinolate | 15.00 |
| Butylated Hydroxytoluene | 0.10 |
| Polyethylene | 2.00 |
| Limnanthes Alba Seed oil | 10.00 |
| Beeswax | 6.80 |
| Candelilla wax | 9.00 |
| Ozokerite | 6.80 |
| C₁₀₋₃₀ Cholesterol/Lanosterol Esters | 6.50 |
| Vitamin E Acetate | 0.50 |

### Transfer Resistant Lipstick

### Procedure

A 8.0-11.0 mg of lipstick was applied to a piece of filter paper. A solution of polyglycerol polymer (Examples 35-48) was applied on top of the filter paper and allowed to dry. After the topcoat dries, a piece of cotton cloth was put onto the filter paper. From the backside of the cotton cloth, a stress is applied for 30 seconds. The cotton cloth was then removed and visually inspected for pigment transfer.

| | Examples | | |
|---|---|---|---|
| Material | 57 | 58 | 59 |
| Chromium Oxide | 8.0 | 8.0 | 8.0 |
| Bis-Octyldodecyl Dimer Dilinoleate/Propanediol | 3.0 | 3.0 | 3.0 |
| Copolymer | | | |
| Avocado Oil | 11.0 | 11.0 | 12.0 |
| Sweet Almond Oil | 14.0 | 11.0 | 15.0 |
| Castor Oil | 22.0 | 22.0 | 22.0 |
| Coconut Oil | 14.0 | 13.0 | 14.0 |
| Carnauba Wax | 11.0 | 11.0 | 11.0 |
| Candelilla Wax | 11.0 | 11.0 | 11.0 |
| Isododecane | 4.0 | 5.0 | 4.0 |
| Examples (35-48) | 2.0 | 5.0 | 0.0 |

### Lipstick Contact Angle

| | Contact Angle | Contact Angle |
|---|---|---|
| Example | H₂O | Olive Oil |
| 59 | 81.45 (+/−1.34) | 24.57 (+/−3.35) |
| 58 | 85.64 (+/−2.97) | 64.02 (+/−1.71) |
| 55 | 66.92 (+/−6.12) | 51.84 (+/−3.71) |
| 46 | 75.80 (+/−2.44) | 56.70 (+/−1.35) |
| 47 | 90.30 (+/−2.73) | 59.14 (+/−0.49) |
| 48 | 96.10 (+/−2.36) | 66.97 (+/−0.48) |

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1.-19. (canceled)

20. A transfer resistant topcoat composition which comprises:
a. a polymer having the following structure; 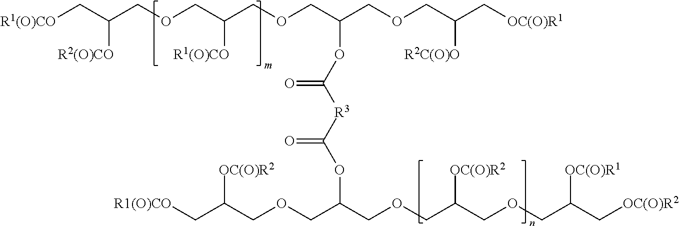
wherein;
R¹ is an alkyl containing 18 to 26 carbons atoms and is solid at 25° C.;
R² is an alkyl containing 8 to 22 carbons atoms and is liquid at 25° C.;
R³ is independently selected from an alkyl containing 7 carbon atoms or an alkyl confining to the following structure: 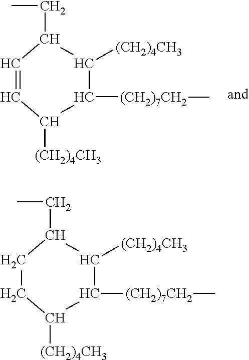
and mixtures thereof with the proviso that at least 50% of R³ is a solid at 25° C.;
n is an integer ranging from 0 to 9;
m is an integer ranging from 0 to 9;
b. a solvent capable of forming a clear solution;
wherein said topcoat composition is applied to the lips after application of a lipstick.

21. The transfer resistant topcoat composition of claim 20 wherein the concentration of polymer ranges from about 2% to 10% by weight and concentration of solvent ranges from about 90% by weight to 98% by weight.

22. The transfer resistant topcoat of claim 20 wherein the polymer has to the following structure: 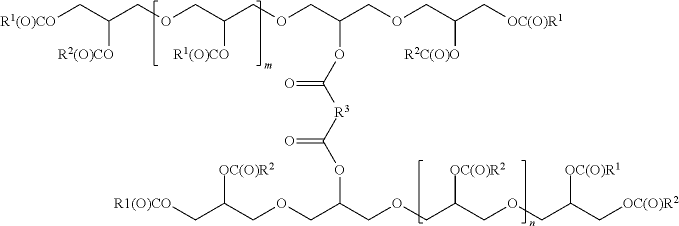
wherein,
R¹ is an alkyl containing 18 carbons atoms and is solid at 25° C.;
R² is an alkyl containing 18 carbons atoms and is liquid at 25° C.;
R³ is an alkyl containing 7 carbon atoms;
n is 0;
m is 0.

23. The transfer resistant topcoat of claim 20 wherein c is 2.

24. The transfer resistant topcoat of claim 20 wherein c is 7.

25. The transfer resistant topcoat of claim 20 wherein c is 8.

26. The transfer resistant topcoat of claim 20 wherein c is 10.

27. The transfer resistant topcoat of claim 20 wherein the solvent is ethanol.

28. The transfer resistant topcoat of claim 20 wherein the solvent is isododecane.

29. The transfer resistant topcoat of claim 20 wherein the solvent is isohexadecane.

30. The transfer resistant topcoat of claim 20 wherein the solvent is propylene glycol n-butyl ether.

31. The transfer resistant topcoat of claim 20 wherein the solvent is ethyl 3-ethoxyproprionate.

32. The transfer resistant topcoat of claim 20 wherein the solvent is propylene glycol methyl ether acetate.

33. The transfer resistant topcoat of claim 20 wherein the solvent is isoparaffin C11-C13.

34. A process for minimizing the transfer of lipstick from lips to which lipstick has been applied which comprises application of a transfer resistant topcoat composition which comprises:
a. a polymer having the following structure; 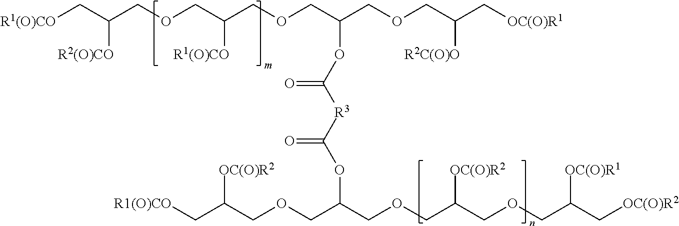
wherein;
R¹ is an alkyl containing 18 to 26 carbons atoms and is solid at 25° C.;
R² is an alkyl containing 8 to 22 carbons atoms and is liquid at 25° C.;
R³ is independently selected from an alkyl containing 7 carbon atoms or an alkyl confining to the following structure: 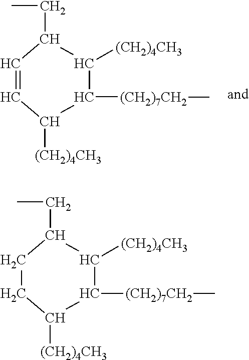
and mixtures thereof with the proviso that at least 50% of R³ is a solid at 25° C.;
n is an integer ranging from 0 to 9;
m is an integer ranging from 0 to 9;
b. a solvent;
wherein said topcoat composition is applied to the lips after application of a lipstick.

35. The process of claim 34 wherein c is 2.

36. The process of claim 34 wherein c is 7.

37. The process of claim 34 wherein c is 8.

38. The process of claim 34 wherein c is 10.
